(19) United States
(12) Patent Application Publication
(10) Pub. No.: US 2025/0144007 A1
(43) Pub. Date: May 8, 2025

(54) COSMETIC COMPOSITION FOR IMPROVING SCALP CONTAINING MICROBIAL FERMENTATION PRODUCT AS ACTIVE INGREDIENT, AND METHOD FOR PRODUCING THE SAME

(71) Applicant: LABIO CO., LTD., Seoul (KR)

(72) Inventors: Ju A KIM, Seoul (KR); Chae Yeon KIM, Seoul (KR); Eun Kyung RYU, Seoul (KR); Hee Sang HAN, Seoul (KR); Hyun Sook LEE, Seoul (KR); Jin Mo KIM, Seoul (KR); Jin Young KIM, Seoul (KR); Ho Sam KI, Seoul (KR); Yang Gyu CHOI, Seoul (KR)

(73) Assignee: LABIO CO., LTD., Seoul (KR)

(21) Appl. No.: 19/019,094

(22) Filed: Jan. 13, 2025

Related U.S. Application Data
(63) Continuation of application No. PCT/KR2023/009785, filed on Jul. 10, 2023.

(30) Foreign Application Priority Data
Jul. 14, 2022 (KR) 10-2022-0086705

(51) Int. Cl. A61K 8/99; A61K 8/44; A61K 8/49; A61K 8/67; A61Q 19/00

(52) U.S. Cl. CPC A61K 8/99; A61K 8/44; A61K 8/4913; A61K 8/678; A61Q 19/00; A61K 2800/85

(57) ABSTRACT

The present invention relates to a microorganism-based cosmetic composition containing, as an active ingredient, at least one of a fermentation product of Lactococcus lactis, a fermentation product of Limosilactobacillus reuteri, and a fermentation product of Saccharomyces cerevisiae, and containing a fermentation product of Lactiplantibacillus plantarum as an active ingredient, as well as a method for producing the same.

### CROSS-REFERENCE TO RELATED APPLICATION

of the PCT This application is a Continuation Application No. PCT/KR2023/009785, filed on Jul. 10, 2023, and claims the benefit of priority from the prior Korean Patent Application No. 10-2022-0086705, filed on Jul. 14, 2022, the disclosures of which are incorporated herein by reference in its entirety.

### TECHNICAL FIELD

The present invention relates to a cosmetic composition containing, as an active ingredient, a microbial fermentation product beneficial for the scalp, and a method for producing the same.

### BACKGROUND ART

Microbiome is a compound word of “microbe” and “biome”, and refers to the microorganisms living in the human body and their genetic information. The number of human microbiomes is more than twice that of pure human cells, and the number of genes thereof is more than 100 times that of pure human cells. Therefore, the human microbiome is also called the second genome because it is impossible to discuss human genes without microbes.

The microbiome is a field that may be widely used in developing new drugs and research on incurable diseases, as it makes it possible to analyze the principles by which beneficial and harmful bacteria are produced and the relationship between diseases. The microbiome is also used to develop foods, cosmetics, and therapeutic agents.

Specifically, microorganisms, the smallest living organisms, produce proteins that directly affect the human body by forming metabolites. There have been cases where intestinal bacteria are analyzed, and intestinal microorganisms extracted from healthy feces are used for therapeutic purposes in other patients in need, cases where blood microorganisms are used to predict premature birth, and cases where they are used to treat obesity.

As research on the microbiome has been continuously and actively conducted, its application areas have gradually expanded.

### DISCLOSURE

### Technical Problem

The present invention is intended to apply microbiome to the scalp and to provide a cosmetic composition containing a microbial fermentation product useful for improving the scalp, and a method for producing the same.

Specifically, the present invention provides a cosmetic composition using microorganisms such as a fermentation product of Lactococcus lactis, a fermentation product of Limosilactobacillus reuteri, a fermentation product of Saccharomyces cerevisiae, and a fermentation product of Lactiplantibacillus plantarum, and a method for producing the same.

### Technical Solution

The present invention provides a microorganism-based cosmetic composition containing, as an active ingredient, at least one of the fermentation products of Lactococcus lactis, a fermentation product of Limosilactobacillus reuteri, and a fermentation product of Saccharomyces cerevisiae.

The present invention provides a microorganism-based cosmetic composition that further contains a fermentation product of Saccharomyces cerevisiae as an active ingredient.

The present invention provides a cosmetic composition further containing a fermentation product of Lactiplantibacillus plantarum as an effective ingredient.

The present invention provides the microorganism-based cosmetic composition wherein the active ingredient comprises arginine, tocopherol, and proline.

The present invention provides the microorganism-based cosmetic composition, which may reduce scalp redness using the active ingredient.

The present invention provides the microorganism-based cosmetic composition that may remove scalp dead skin cells from the scalp using the active ingredient.

The present invention provides the microorganism-based cosmetic composition, which may improve scalp pH using the active ingredient.

The present invention provides the microorganism-based cosmetic composition, which may improve scalp elasticity using the active ingredient.

The present invention provides the microorganism-based cosmetic composition, which may improve the antioxidant activity of scalp dead skin cells using the active ingredient.

The present invention provides the microorganism-based cosmetic composition, which may alleviate scalp itching using the active ingredient.

The present invention provides a method for producing a microorganism-based cosmetic composition, comprising: step (a) of preparing a culture medium containing peptone, yeast, and a carbon source; step (b) of producing a microbial fermentation product by culturing any one or more of Lactococcus lactis, Limosilactobacillus reuteri, Saccharomyces cerevisiae, and Lactiplantibacillus plantarum in the culture medium; and step (c) of mixing the culture medium and the microbial fermentation product, followed by culturing, wherein the microorganism-based cosmetic composition contains, as an active ingredient, a fermentation product of Lactococcus lactis, a fermentation product of Limosilactobacillus reuteri, and a fermentation product of Saccharomyces cerevisiae, and contains a fermentation product of Lactiplantibacillus plantarum as an active ingredient.

The present invention provides the method for producing the microorganism-based cosmetic composition, wherein the culture medium in step (a) further contains sodium chloride or magnesium phosphate.

The present invention provides the method for producing the microorganism-based cosmetic composition, wherein the culturing in step (c) is performed for 30 hours or more.

The present invention provides the method for producing the microorganism-based cosmetic composition, wherein the culture medium for Saccharomyces cerevisiae contains yeast at a concentration of 3 to 10 g/L and the carbon source at a concentration of 5 to 15 g/L.

The present invention provides the method for producing the microorganism-based cosmetic composition, wherein the culture medium for Lactiplantibacillus plantarum contains yeast at a concentration of 10 to 30 g/L and the carbon source at a concentration of 1 to 3 g/L.

The present invention provides the method for producing the microorganism-based cosmetic composition, wherein the culture medium and the microbial fermentation product are mixed at a volume ratio of 1:8 to 1:12.

The present invention provides a microorganism-based cosmetic composition produced by the production method.

### Advantageous Effects

The microorganism-based cosmetic composition according to the present invention has excellent effects of improving scalp pH, exhibiting anti-inflammatory function, reducing scalp redness, improving scalp elasticity, removing scalp dead skin cells, and alleviating scalp itching.

In addition, the microorganism-based cosmetic composition according to the present invention has an excellent effect in improving the balance of the scalp microbiome.

### BRIEF DESCRIPTION OF DRAWINGS

FIG. 1 shows a growth curve as a function of culture time in the medium of the present invention.

FIG. 2 shows the results of an experiment conducted to evaluate the anti-inflammatory effects of Lactococcus lactis, Limosilactobacillus reuteri, Saccharomyces cerevisiae, and Lactiplantibacillus plantarum in the present invention.

FIG. 3 shows the results of an experiment conducted to evaluate the anti-inflammatory effects of 5% arginine and proline, which are the metabolites of the strains used in the present invention.

FIG. 4 shows the reduction of the inflammation-related gene IL-1β in keratinocytes in the present invention.

FIG. 5 is a graph showing the improvement rate obtained by applying the metabolites of the strains used in the present invention to the human body and measuring the scalp surface area using a chromameter.

FIG. 6 shows photographs taken before and after applying the metabolite of the strains used in the present invention to the human body.

FIG. 7 is a graph showing the results of an experiment conducted to evaluate the reduction rate of scalp dead skin cells after applying the metabolites of the strains used in the present invention to the human body.

FIG. 8 depicts photographs showing the state of dead skin cells on the scalp surface after applying the metabolites of the strains used in the present invention to the human body.

FIG. 9 is a graph showing the results of an experiment conducted to measure scalp pH after applying the metabolites of the strains used in the present invention to the human body.

FIG. 10 is a graph showing the results of an experiment conducted to evaluate the improvement rate of scalp pH after applying the metabolites of the strains used in the present invention to the human body.

FIG. 11 is a graph showing the results of an experiment conducted to measure scalp elasticity after applying the metabolites of the strains used in the present invention to the human body.

FIG. 12 is a graph showing the results of an experiment conducted to measure the antioxidant activity value of scalp dead skin cells after applying the metabolites of the strains used in the present invention to the human body.

FIG. 13 is a graph showing the results of an experiment conducted to measure scalp itching after applying the metabolites of the strains used in the present invention to the human body.

FIG. 14 is a graph showing the results of an experiment conducted to measure the difference in scalp microbiome between before and after applying the culture medium and the metabolites of the strains used in the present invention to the human body.

FIG. 15 is a graph showing experimental results indicating a diversity relative to the diversity of all microbial species, which is taken as 1, after applying the metabolites of the strains used in the present invention to the scalp.

### MODE FOR INVENTION

The terms used in this specification have been chosen, as far as possible, from commonly used general terms, taking into account the functions of the present invention. However, these terms may vary depending on the intent of those skilled in the art, court the emergence of new technologies.

In certain cases, terms may have been arbitrarily selected by the applicant. In such cases, their meanings will be described in detail in the relevant sections of the invention description. Therefore, the terms used in the present invention should be defined not by their simple nomenclature but based on their meanings and the overall content of the present invention.

Unless otherwise defined, all terms used herein, including technical and scientific terms, have the same meanings as commonly understood by those of ordinary skill in the art to which the present invention pertains. Terms defined in general dictionaries should be interpreted as having meanings consistent with the context of the relevant technology and should not be interpreted in an overly idealized or excessively formal sense unless explicitly defined in this application.

Numerical ranges include the values defined within the ranges. All maximum numerical limits given throughout this specification include all lower numerical limits as if explicitly stated. All minimum numerical limits given throughout this specification include all higher numerical limits as if explicitly stated. All numerical limits given in this specification include all better numerical ranges within the broader range as if a narrower range were explicitly stated.

Hereinafter, embodiments of the present invention will be described in detail, but it is obvious that the present invention is not limited to the following embodiments.

The present invention provides a cosmetic composition based on a fermentation product of Lactococcus lactis, a fermentation product of Limosilactobacillus reuteri, a fermentation product of Saccharomyces cerevisiae, and a fermentation product of Lactiplantibacillus plantarum, and a method for producing the same.

In one embodiment, the present invention provides a microorganism-based cosmetic composition containing, as an active ingredient, any one or more of a fermentation product of Lactococcus lactis, a fermentation product of Limosilactobacillus reuteri, a fermentation product of Saccharomyces cerevisiae, and a fermentation product of Lactiplantibacillus plantarum.

The fermentation product is produced under specific culture conditions. In one embodiment, the fermentation product means one obtained by culturing in a culture medium containing distilled water, peptone or tryptone, yeast (extract), and a carbon source. The yeast (nitrogen source) and the carbon source are major nutrients for microorganisms. The carbon source may comprise either a reducing sugar or a non-reducing sugar, and specifically, it may comprise glucose, fructose, maltose, galactose, saccharose, etc.

In one embodiment, the present invention relates to a cosmetic composition that is used on the scalp, wherein the cosmetic composition may be used as a cosmetic composition for reducing scalp redness using the active ingredient, a cosmetic composition for removing scalp dead skin cells using the active ingredient, a cosmetic composition for improving scalp pH using the active ingredient, a cosmetic composition for improving scalp elasticity using the active ingredient, cosmetic composition for improving the antioxidant activity of scalp dead skin cells using the active ingredient, or cosmetic composition for alleviating scalp itching using the active ingredient.

“Reducing scalp redness” may mean reducing or eliminating redness of the scalp, thereby keeping the scalp healthy.

“Reducing scalp dead skin cells” may mean effectively removing dead skin cells accumulated on the scalp or suppressing the production thereof, thereby keeping the scalp healthy.

“Improving scalp pH” may mean maintaining or normalizing the pH balance of the scalp, thereby creating a healthy scalp environment.

“Improving scalp elasticity” may mean improving the flexibility and elasticity of the scalp, thereby creating a healthy and active scalp environment.

“Improving the antioxidant activity of scalp dead skin cells” may mean reducing oxidative stress occurring in the stratum corneum of the scalp and preventing aging, thereby protecting the scalp and maintaining the scalp in a healthy state.

“Alleviating scalp itching” may mean that the scalp is supplied with adequate moisture and nutrients, and thus is maintained in a soft and flexible state and exhibits glossy appearance properties.

The fermentation product of Lactococcus lactis, the fermentation product of Limosilactobacillus reuteri, the fermentation product of Saccharomyces cerevisiae, and the fermentation product of Lactiplantibacillus plantarum, which are used in the present invention, comprise produce arginine, tocopherol, and proline. The arginine is a type of amino acid that increases blood flow and assists in blood vessel formation and growth, thereby exhibiting anti-inflammation and skin immunity functions. Tocopherol is vitamin E that has antioxidant, anti-inflammatory, and pain relief functions, and has the function of preventing oxidation of essential fatty acids and inhibiting skin aging. In addition, the proline is a type of amino acid that is produced in some parts of the body, may be obtained through the intake of protein foods, and is involved in the formation of collagen, thereby exhibiting the function of preventing aging and repairing damaged skin.

The present invention provides a method for producing a microorganism-based cosmetic composition.

In one embodiment, the present: invention provides a method for producing a microorganism-based cosmetic composition, comprising: step (a) of preparing a culture medium containing peptone, yeast, and a carbon source; step (b) of producing a microbial fermentation product by culturing any one or more of Lactococcus lactis, Limosilactobacillus reuteri, Saccharomyces cerevisiae, and Lactiplantibacillus plantarum in the culture medium; and step (c) of mixing the culture medium and the microbial fermentation product, followed by culturing, wherein the microorganism-based cosmetic composition contains, as an active ingredient, a fermentation product of Lactococcus lactis, a fermentation product of Limosilactobacillus reuteri, and a fermentation product of Saccharomyces cerevisiae, and product contains a fermentation of Lactiplantibacillus plantarum as an active ingredient.

In one embodiment of the present invention, the culture medium in step (a) may further contain sodium chloride or magnesium phosphate. In another embodiment, when Lactococcus lactis, Limosilactobacillus reuteri, or Saccharomyces cerevisiae is used, the culture medium may further contain magnesium phosphate. When Lactiplantibacillus plantarum is used, the culture medium may further contain sodium chloride. In another embodiment, step (a) may be performed at 100° C. or higher, 100 to 150° C., 100 to 130° C., 110 to 130° C., or 115 to 130° C. In another embodiment, step (a) may be performed at 0.5 to 3 atm, 1 to 3 atm, 1 to 2.5 atm, or 1 to 2 atm. In another embodiment, step (a) may comprise culturing for 10 minutes or more, followed by sterilization.

In the present invention, step (b) comprise adding at least one of Lactococcus lactis, Limosilactobacillus reuteri, Saccharomyces cerevisiae, and Lactiplantibacillus plantarum in the culture medium or medium composition of step (a), thereby producing a fermentation product.

In one embodiment, a single colony of at least one of Lactococcus lactis, Limosilactobacillus reuteri, Saccharomyces cerevisiae, and Lactiplantibacillus plantarum may be inoculated and cultured. Preferably, the culturing may be performed at a temperature of 30 to 40° C. for 12 hours or more.

In one embodiment of the present invention, when Lactococcus lactis, Limosilactobacillus reuteri, or Saccharomyces cerevisiae is used, the culture medium or medium composition may contain yeast at a concentration of 3 to 10 g/L, specifically 4 to 6 g/L, more specifically 4.5 to 5.5 g/L. In another embodiment, when Lactococcus lactis, Limosilactobacillus reuteri, or Saccharomyces cerevisiae is used, the culture medium or medium composition may contain the carbon source at a concentration of 5 to 15 g/L, specifically 8 to 12 g/L, more specifically 9.5 to 10.5 g/L.

In one embodiment, when Lactiplantibacillus plantarum is used, the culture medium or medium composition may contain yeast at a concentration of 0.05 to 1 g/L, specifically 0.1 to 0.5 g/L, more specifically 0.2 to 0.3 g/L. In another embodiment, when Lactiplantibacillus plantarum is used, the culture medium or medium composition may contain the carbon source at a concentration of 0.01 to 0.5 g/L, specifically 0.1 to 0.3 g/L, more specifically 0.2 to 0.25 g/L.

In the present invention, step (c) may comprise mixing the culture medium and the microbial fermentation product at a volume ratio of 1:8 to 1:12, and at a volume ratio of 1:10 in a specific embodiment. After the mixing, the mixture may be cultured for 24 hours or more, and the entire culture may be collected. After centrifugation, the supernatant may be collected. In a specific embodiment, the culture time after the mixing is preferably 30 hours or more when Lactococcus lactis, Limosilactobacillus reuteri, or Saccharomyces cerevisiae is used, and the culture time after the mixing is preferably 50 hours or more when Lactiplantibacillus plantarum is used.

In one embodiment, the present invention may provide a microorganism-based cosmetic composition produced by the above-described production method. In addition, the present invention may produce a product created using the cosmetic composition according to the present invention.

In one embodiment, the present invention may provide a microorganism-based cosmetic composition produced by the above-described production method. In addition, the present invention may produce a product created using the cosmetic composition according to the present invention.

The above cosmetic composition may contain various additives such as an emulsifier, a preservative, a diluent, and a pigment, in consideration of the function or intended use of the composition.

The cosmetic composition may further contain an agent component that may be combined with common external preparations for the scalp and hair. Specific examples of the agent component include solubilizing agents, surfactants, moisturizers, thickeners, pH-adjusting agents, preservatives, antioxidants, metal ion-sequestering agents, germicides, anti-inflammatory agents, antimicrobial agents, solvents, coloring agents, fragrance ingredients, or the like.

The solubilizing agents may include isopropyl myristate, polyethylene glycol, medium-chain fatty acid triglycerides, hydrocarbons, glycols, and the like.

Among the surfactants, anionic surfactants may include ammonium lauryl sulfosuccinate, ammonium lauryl sulfate, sodium cocoyl isethionate, sodium lauryl isethionate, sodium lauryl sulfate, triethanolamine lauryl sulfate, sodium lauryl ether sulfate (1 to 3 ethylene oxides), and the like. Nonionic surfactants may include polyoxyethylene alkyl ethers, polyoxyethylene fatty acid esters, polyoxyethylene, hydrogenated castor oil derivatives, fatty acid diethanolamides, glyceryl stearate, and the like. Cationic surfactants may include tertiary aliphatic amine salts, alkyl trimethylammonium chloride, dialkyl dimethylammonium chloride, and the like. Amphoteric surfactants may include betaine, betaine amide, sulfobetaine, steartrimonium chloride, and the like.

The moisturizers may include glycerin, propylene glycol, 1,3-butylene glycol, dipropylene glycol, sorbitol, and the like.

The thickeners may include water-soluble polymeric compounds such as methylcellulose, hydroxyethyl cellulose, carrageenan, carboxymethyl cellulose, and hydroxymethyl cellulose.

Specific examples of pH-adjusting agents include citric acid, sodium hydroxide, triethanolamine, sodium citrate, phosphoric acid, sodium phosphates, lactic acid, and the like.

The preservatives may include hexanediol, benzoic acid, ρ-hydroxybenzoic acid esters, a mixture of methylchloroisothiazolinone and methylisothiazolinone, phenoxyethanol, DMDM hydantoin, and the like. The antioxidants may include dibutyl hydroxytoluene, ascorbic acid, and the like.

The metal ion-sequestering agents may include disodium ethylenediaminetetraacetate, tetrasodium ethylenediaminetetraacetate, and the like. The germicides may include chlorhexidine gluconate, quaternary ammonium salts, piroctone olamine, zinc pyrithione suspension, iodopropynyl butylcarbamate, salicylic acid, and the like.

The anti-inflammatory agents may include monoammonium glycyrrhizinate, dipotassium glycyrrhizinate, stearyl glycyrrhizinate, camomile, alpha-bisabolol, allantoin, and the like. Specific examples of the antimicrobial agents may include phenoxyethanol, chlorhexidine, chlorhexidine gluconate, piroctone olamine, ketoconazole, arnica extracts, iodopropynyl butylcarbamate, benzalkonium chloride, benzethonium chloride, benzoic acid and salts thereof, benzyl alcohol, lavender, rosemary, salicylic acid, triclocarban, zinc pyrithione suspension, and the like.

The solvents may include ethanol, purified water, Tween 20, cyclomethicone, mineral oil, dimethicone, and the like. The coloring agents and fragrance ingredients may include conventional agents used in scalp and hair preparations.

In addition, the composition may be formulated into a hair treatment composition such as an emulsion, cream, paste, gel, face lotion, pack, lotion, powder, spray, soap, or the like. Specifically, the composition may be formulated into one or more selected from the group consisting of hair toner, hair lotion, hair cream, hairspray, hair mousse, hair gel, hair soap, hair shampoo, hair rinse, hair pack, and hair treatment. Specifically, it may be formulated into liquid hair shampoo.

In the formulation, ingredients other than the above-mentioned essential ingredients may be appropriately selected and blended by those skilled in the art depending on the intended use or purpose of an external preparation comprising the composition of the present invention.

For example, when the formulation of the composition is a paste, cream or gel, animal fibers, plant fibers, wax, paraffin, starch, tragacanth, cellulose derivatives, polyethylene glycol, silicone, bentonite, silica, talc or zinc oxide may be used as a carrier ingredient.

When the formulation of the composition is powder or spray, lactose, talc, silica, aluminum hydroxide, calcium silicate, or polyamide powder may be used as a carrier ingredient. In particular, the composition may further contain a propellant such as chlorofluorocarbon, propane, butane, or dimethyl ether.

When the formulation of the composition is a solution or emulsion, a solvent or a solubilizing or emulsifying agent may be used as a carrier ingredient. For example, water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, or fatty acid esters of sorbitan may be used.

When the formulation of the composition is a suspension, liquid diluents such as water, ethanol, and propylene glycol, suspending agents such as ethoxylated isostearyl alcohols, polyoxyethylene sorbitol esters, and polyoxyethylene sorbitan esters, microcrystalline aluminum metahydroxide, bentonite, agar, or tragacanth may be used as a carrier ingredient

The present invention will be further described by way of the following examples, but it is obvious that the present invention is not limited by the following examples.

### Preparation Example 1. Culture of Lactococcus lactis Microorganism

For a fermentation product of Lactococcus lactis, a medium having a composition containing 10 g/L sucrose, 4.5 g/L peptone, 10 g/L yeast extract, 28 g/L potassium phosphate, 2 g/L sodium chloride, and 0.2 g/L magnesium phosphate 0.2 g/L was prepared and sterilized under conditions of 121° C. or higher and 1.5 atm.

Lactococcus lactis inoculated into the sterilized medium and cultured with shaking at 25 to 40° C. for 48 hours or more.

The sterilized medium and the shaking culture were mixed with each other and then started to be cultured under conditions of 1 vvm air, 200 rpm, 37° C., and initial pH 7.0.

The D.O. was maintained at 70%, the temperature was maintained at 37° C., and the remaining conditions can be changed to control the D.O.

After 36 hours, the culture was terminated, and the entire culture was collected and centrifuged. The supernatant was collected and filtered after centrifugation.

### Preparation Example 2. Culture of Limosilactobacillus reuteri Microorganism

For a fermentation product of Limosilactobacillus reuteri, a medium having a composition containing 20 g/L yeast extract, 20 g/L glucose, 7 g/L sodium acetate, 1 g/L ammonium citrate, 0.2 g/L magnesium phosphate, and 0.23 g/L manganese sulfate was prepared and sterilized under conditions of 121° C. or higher and 1.5 atm.

Limosilactobacillus reuteri was inoculated into the sterilized medium and cultured with shaking at 25 to 40° C. for 48 hours or more.

The sterilized medium and the shaking culture were mixed with each other and then started to be cultured under conditions of 1 vvm air, 200 rpm, 37° C., and initial pH 7.0.

The D.O. was maintained at 70, the temperature was maintained at 37° C., and the remaining conditions can be changed to control the D.O.

After 36 hours, the culture was terminated, and the entire culture was collected and centrifuged. After centrifugation, the obtained supernatant was collected and filtered.

### Preparation Example 3. Culture of Saccharomyces cerevisiae Microorganism

For a fermentation product of Saccharomyces cerevisiae, a medium having a composition containing 3 g/L yeast extract, 3 g/L malt extract, 5 g/L peptone, and 10 g/L dextrose was prepared and sterilized under conditions of 121° C. or higher, and 1.5 atm.

Saccharomyces cerevisiae was inoculated into the sterilized medium and cultured with shaking at 25 to 40° C. for 48 hours or more.

The sterilized medium and the shaking culture were mixed with each other and then started to be cultured under conditions of 1 vvm air, 200 rpm, 37° C., and initial pH 7.0.

The D.O. was maintained at 70, the temperature was maintained at 37° C., and the remaining conditions can be changed to control the D.O.

After 36 hours, the culture was terminated, and the entire culture was collected and centrifuged. After centrifugation, the obtained supernatant was collected and filtered.

### Preparation Example 4. Culture of Lactiplantibacillus plantarum Microorganism

A medium having a composition containing 20 g/L yeast extract, 2 g/L peptone, 0.8 g/L magnesium phosphate, and 15 g/L glucose was prepared and sterilized under conditions of 121° C. or higher and 1.5 atm.

Lactiplantibacillus plantarum was inoculated into the sterilized medium and cultured with shaking at 30 to 40° C. for 48 hours or more.

The sterilized medium and the shaking culture were mixed with each other and then started to be cultured under conditions of 1 vvm air, 200 rpm, 37° C., and initial pH 7.0.

The sterilized medium and the shaking culture were mixed with each other and then started to be cultured under conditions of 1 vvm air, 200 rpm, 37° C., and initial pH 7.0.

The D.O. was maintained at 70, the temperature was maintained at 37° C., and the remaining conditions can be changed to control the D.O.

After 60 hours, the culture was terminated, and the entire culture was collected and centrifuged. After centrifugation, the obtained supernatant was collected and filtered.

### Preparation Example 5. Process of Producing Cosmetic Compositions

The final products were produced by adding 1,2-hexanediol to the cultures of the Preparation Examples.

### Experimental Example 1: Experiment for Medium Optimization

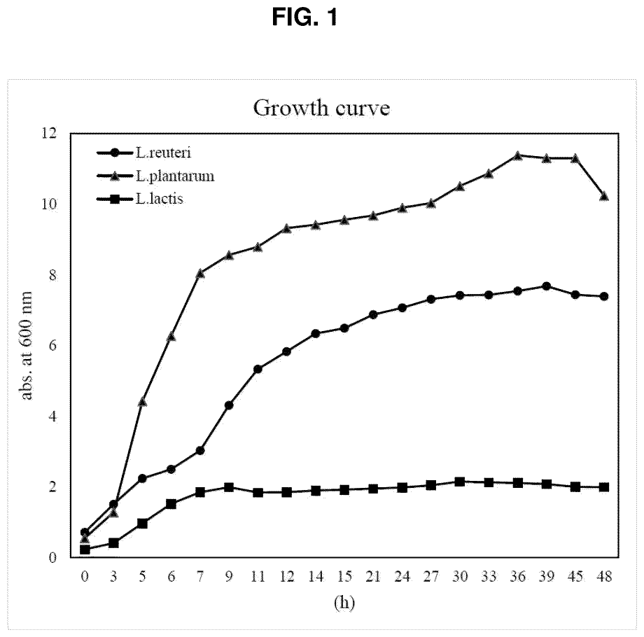

### 1) Culture Time (FIG. 1)

Lactococcus lactis, Limosilactobacillus reuteri, Saccharomyces cerevisiae, or Lactiplantibacillus plantarum was added to the finally selected medium composition.

Then, to optimize the culture conditions, samples were taken every 3 hours, and growth was measured using a spectrophotometer at 600 nm.

Referring to FIG. 1, the highest growth was observed at a time point of 36 hours or more.

### Experimental Example 2. In Vitro Experiments

### 1) Experiment on the Anti-Inflammatory Effect of Composition by Measurement of Nitric Oxide Production

RAW 264.7 cells were seeded at 2.5×10⁵ cells/well and cultured at 37° C. under 5% CO₂ for 24 hours. After culture, the cells were starved with serum-free medium, and treated with a sample of each of strain extracts (Lactococcus lactis, Limosilactobacillus reuteri, Saccharomyces cerevisiae, and Lactiplantibacillus plantarum) diluted at varying concentrations.

Meanwhile, the cells were treated with 20 μg/ml of dexamethasone as a positive control and cultured under conditions of 37° C. and 5% CO₂ for 1 hour. Next, the cells were treated with 1 μg/ml of LPS and cultured at 37° C. under 5% CO₂ for 24 hours.

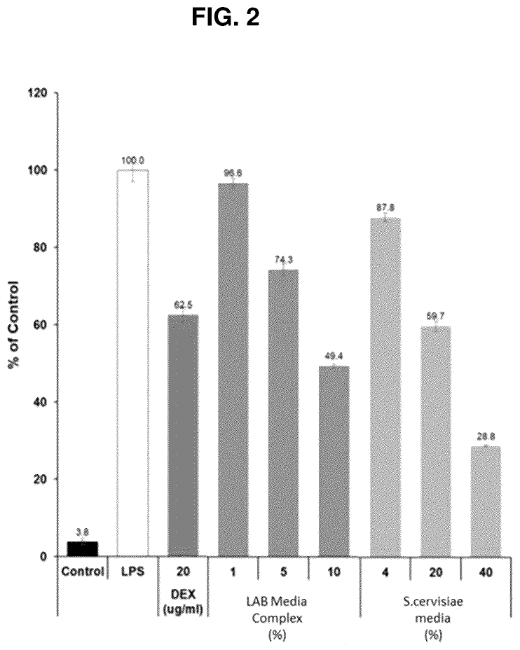

100 μl of the medium containing the synthesized NO was transferred to a 96-well plate, and 500 μM NaNO₂ was used standard. The 96-well plate was treated with each as a concentration (50 to 0 μM) and incubated at room temperature for 20 minutes, and then the absorbance was measured at 540 nm. The value of each synthesized NO was corrected using a standard curve, and the values were averaged. The results are shown in FIG. 2.

In order to evaluate the anti-inflammatory effect of the cosmetic composition, RAW264.7 cells were treated with each test substance at various concentrations in a non-cytotoxic range, and the anti-inflammatory effect of the test substance was evaluated by measuring the amount of nitric oxide production.

As a result of evaluating the anti-inflammatory effect of the composition of the present invention by measurement of nitric oxide production, it was confirmed that NO production was inhibited by 51% upon treatment with 10% Lactiplantibacillus plantarum extract inhibited, and NO production was inhibited by 41% upon treatment with 20% Saccharomyces cerevisiae extract, indicating that these strain extracts have excellent anti-inflammatory effects.

### 2) Experiment on Anti-Inflammatory Effects of Strain Metabolites by Measurement of Nitric Oxide Production

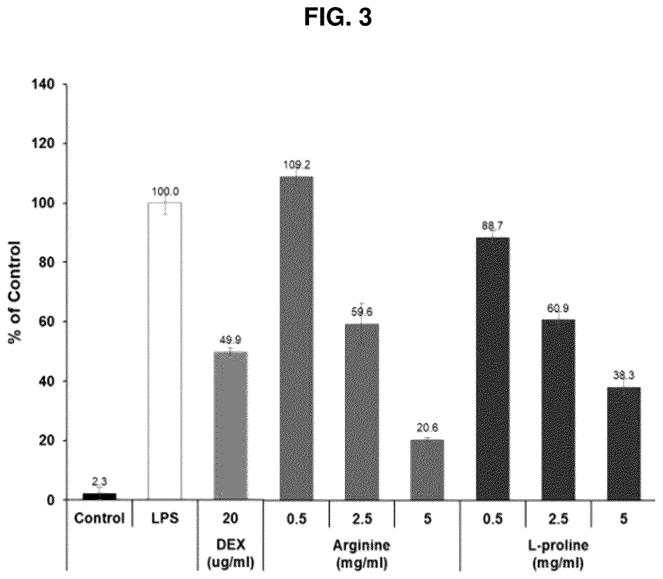

The anti-inflammatory effects of arginine and proline, which are metabolites obtained from the culture of each of Lactococcus lactis, Limosilactobacillus reuteri, Saccharomyces cerevisiae, and Lactiplantibacillus plantarum, were evaluated according to the above-described method, and the results are shown in FIG. 3.

It was confirmed that NO production was inhibited by 40% upon treatment with 2.5 mg/ml of arginine, and NO production was inhibited by 40% upon treatment with 2.5 mg/ml of proline. It was also confirmed that the metabolites obtained from each culture medium had excellent anti-inflammatory effects.

### 3) Experiment on Reduction of Inflammation-Related Gene IL-1β in Keratinocytes

HaCaT cells were seeded into a 6-well plate at a density of 1.0×10⁶ cells/well and cultured at 37° C. under 5% CO₂ for 24 hours.

After culturing, the cells were starved with serum-free medium, treated with 40% cell lysate mixture (stimulus) of Cutibacterium acnes and Staphylococcus aureus, which are scalp inflammation-causing bacteria, and Malassezia furfur, which is scalp dandruff-causing bacteria, and cultured at 37° C. under 5% CO₂ for 24 hours.

Thereafter, the cells were treated with a sample of a mixture of strain extracts (Lactococcus lactis, Limosilactobacillus reuteri, Saccharomyces cerevisiae, and Lactiplantibacillus plantarum), which has been diluted at varying concentrations.

Meanwhile, the cells were 10 μg/ml of ketoconazole as a positive control and cultured under 37° C. and 5% CO₂ for 24 hours. RNA was extracted from the cultured cells and synthesized into cDNA, and then the expression level of the IL-1β gene, an inflammation-related gene, was analyzed.

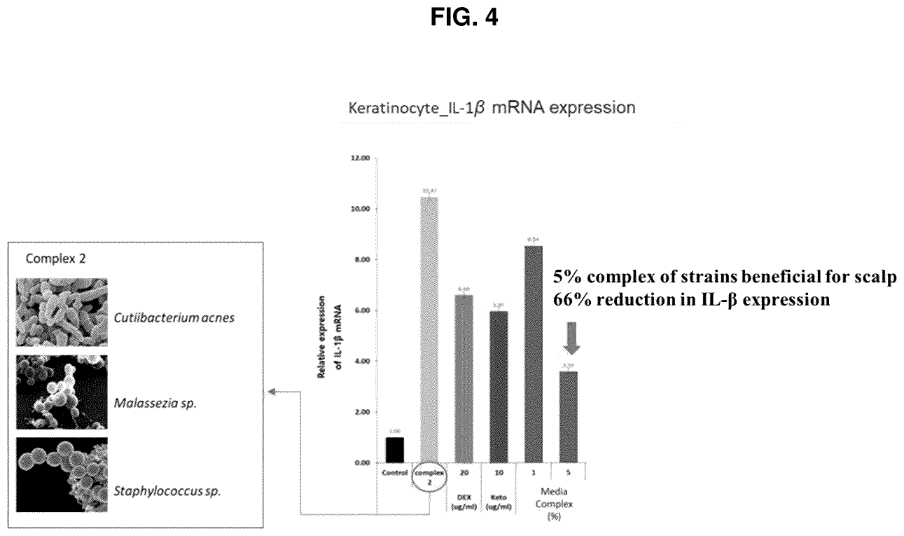

The values were averaged, and the results are shown in FIG. 4.

It was confirmed that the mixture of the strain extracts reduced the expression of the inflammatory gene by 65% or more.

### Experimental Example 3: Human Application Experiments

For human application experiments, Examples and Comparative Examples were set as follows, and the activity of each experimental group was compared and evaluated.

TABLE 1
[Content (parts by weight)]

| | Example | | | | Comparative Example | | | |
|---|---|---|---|---|---|---|---|---|
| | 1 | 2 | 3 | 4 | 1 | 2 | 3 | 4 |
| Lactococcus lactis | 150 | 100 | 100 | 75 | 300 | — | — | — |
| Limosilactobacillus | 150 | 100 | 100 | 75 | — | 300 | — | — |
| reuteri | | | | | | | | |
| Saccharomyces | — | 100 | — | 75 | — | — | 300 | — |
| cerevisiae | | | | | | | | |
| Lactiplantibacillus | — | — | 100 | 75 | — | — | — | 300 |
| plantarum | | | | | | | | |

Referring to Table 1 above, mixtures of different microorganisms mixed at predetermined ratios were used as the Examples.

### 1) Human Application Test for Reduction of Scalp Redness

Each of sample solutions obtained by mixing fermentation product samples prepared under different conditions with purified water was applied to the scalp of an experimental group with scalp redness symptoms three days a week for one month.

After a predetermined amount of the sample solution was applied to a large area of the scalp, it was spread by massaging with the inner part of the fingers. After one month, the degree of scalp redness was analyzed.

To measure the scalp redness, the degree of scalp redness was photographed using Antera 3D. The photographs taken in hemoglobin mode were analyzed for skin redness (a-value) by designating a specific area using Image-Pro Plus, an image analysis program.

TABLE 2

| | Redness reduction rate (%) |
|---|---|
| Example 1 | 29.4 |
| Example 2 | 33.6 |
| Example 3 | 35.1 |
| Example 4 | 37.6 |
| Comparative Example 1 | 18.7 |
| Comparative Example 2 | 21.5 |
| Comparative Example 3 | 17.5 |
| Comparative Example 4 | 19.5 |

Referring to the experimental shown in Table 2 above, the Examples generally showed a higher rate of reduction in scalp redness than the Comparative Examples.

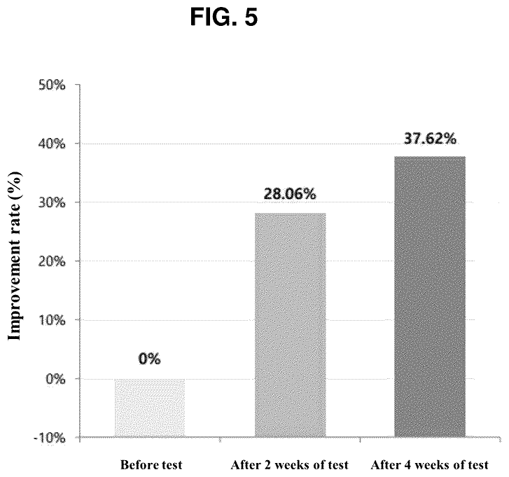

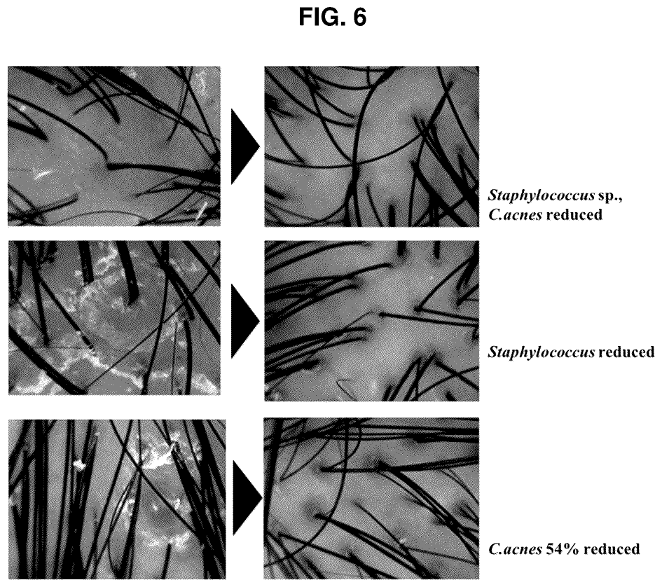

In particular, Example 4 showed the highest reduction rate of 37.6% (FIGS. 5 and 6), which was about 1.9 times higher than that of Comparative Example 4 (19.5%).

The above results suggest that the fermentation product obtained by mixing different microorganisms at a specific ratio is effective in reducing scalp redness.

### 2) Human Application Test for Removal of Scalp Dead Skin Cells

Each of sample solutions obtained by mixing fermentation product samples prepared under different conditions with purified water was applied to the scalp of experimental groups with excessive scalp dead skin cell (dandruff) symptoms three days a week for one month.

After a predetermined amount of the sample solution was widely applied to the scalp, it was spread by massaging it with the inner part of the fingers and washed out after about 20 minutes. After one month, the degree of removal of dead skin cells from the scalp was evaluated.

The degree of scalp dead skin cells was determined by visually evaluating the amount and distribution area of scalp dead skin cells using a scalp scope (Visioscope PC35, Courage-Khazaka, Germany).

TABLE 3

| | Reduction rate (%) of scalp |
|---|---|
| | dead skin cells |
| Example 1 | 12.85 |
| Example 2 | 14.23 |
| Example 3 | 15.32 |
| Example 4 | 17.63 |
| Comparative Example 1 | 7.62 |
| Comparative Example 2 | 6.45 |
| Comparative Example 3 | 8.54 |
| Comparative Example 4 | 7.85 |

Referring to Table 3 above, The Examples generally showed a higher reduction rate in scalp dead skin cells compared to the Comparative Examples.

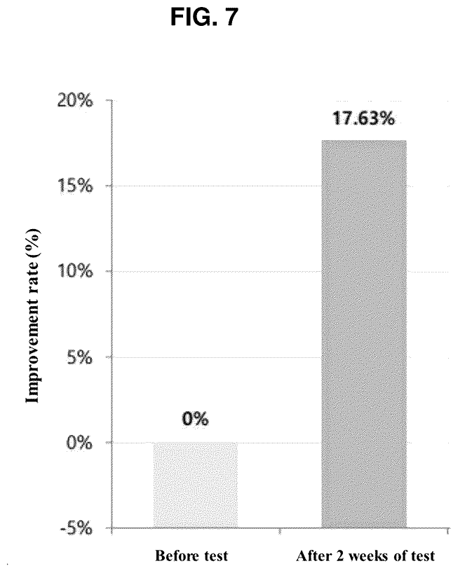

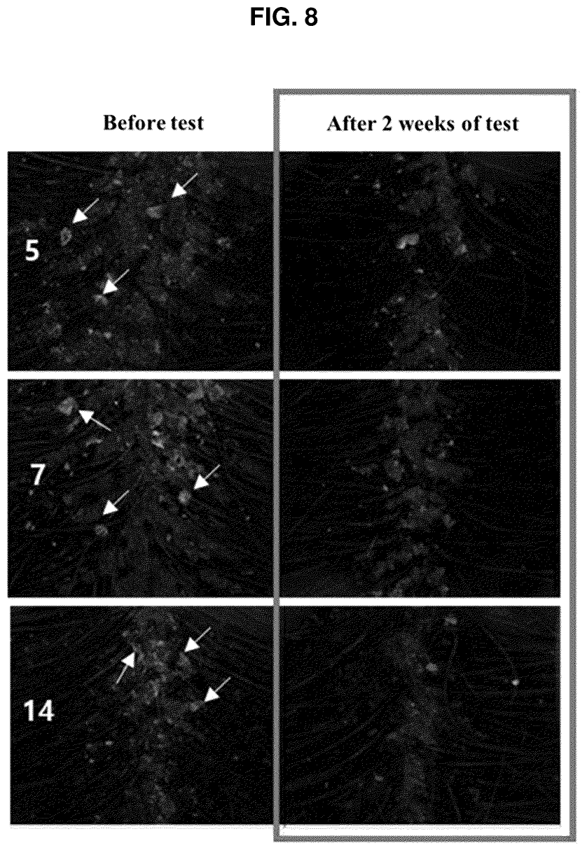

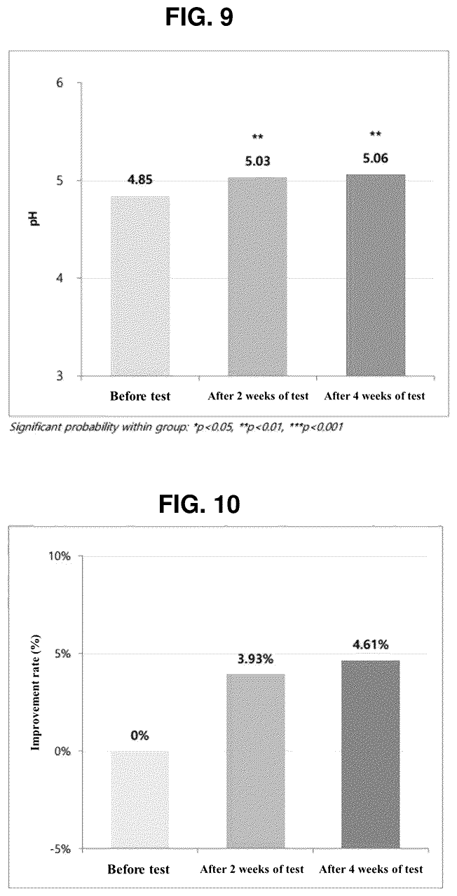

In particular, Example 4 showed the highest reduction rate of 17.63% (FIGS. 7 and 8), which was about 2.7 times higher than that of Comparative Example 2 (6.45%).

The above results suggest that fermentation product samples prepared under different conditions are effective in removing scalp dead skin cells, and in particular, these results show that the specific combination of the microorganisms mixed at the specific ratio had a significant effect on reducing scalp dead skin cells.

### 3) Human Application Test for Improvement in Scalp pH

The sample solution of each of the Examples and the Comparative Examples was applied to the scalp, and after one month, the change in the scalp pH was evaluated.

The scalp pH was measured using a pH meter (PH905, Courage-Khazaka, Germany), and the scalp pH normalization rate of the experimental group was evaluated.

Generally, skin pH increases due to UV radiation, etc. Thus, when the increased pH decreased to the normal range of pH 4 to 6, it was evaluated as normalized.

TABLE 4

| | Scalp pH normalization rate |
|---|---|
| | (%) |
| Example 1 | 3.68 |
| Example 2 | 4.41 |
| Example 3 | 4.32 |
| Example 4 | 4.61 |
| Comparative Example 1 | 1.26 |
| Comparative Example 2 | 1.87 |
| Comparative Example 3 | 1.74 |
| Comparative Example 4 | 1.53 |

Referring to Table 4 above, the Examples generally showed a higher scalp pH normalization rate than the Comparative Examples.

In particular, Example 4 showed the highest normalization rate of 4.61% (FIG. 10), which was about 3.7 times higher than that of Comparative Example 1 (1.26%).

The above results suggest that the fermentation product sample of the Example is effective in normalizing the scalp pH that has increased due to UV radiation, etc.

### 4) Human Application Test for Improvement in Scalp Elasticity

The sample solution of each of the Examples and the Comparative Examples was applied to the scalp, and after one month, the degree of improvement in scalp elasticity was evaluated.

The scalp elasticity at the same central area of the crown of the head was measured before and after the application of the test product using a Ballistometer BLS780 (Dia-Stron, UK), which measures the skin's ability to return to its original shape.

The ballistometer probe was placed in contact with the skin, and three measurements were made and averaged. The average values were used as the evaluation data for skin elasticity. The CoR (Coefficient of Restitution) value was used as the evaluation data for skin elasticity, and a higher CoR value indicates more improved scalp elasticity.

TABLE 5

| | Scalp elasticity improvement |
|---|---|
| | rate (%) |
| Example 1 | 19.54 |
| Example 2 | 23.12 |
| Example 3 | 22.64 |
| Example 4 | 25.74 |
| Comparative Example 1 | 11.74 |
| Comparative Example 2 | 13.25 |
| Comparative Example 3 | 9.65 |
| Comparative Example 4 | 10.64 |

Referring to Table 5 above, the Examples generally showed a higher improvement in scalp elasticity than the Comparative Examples.

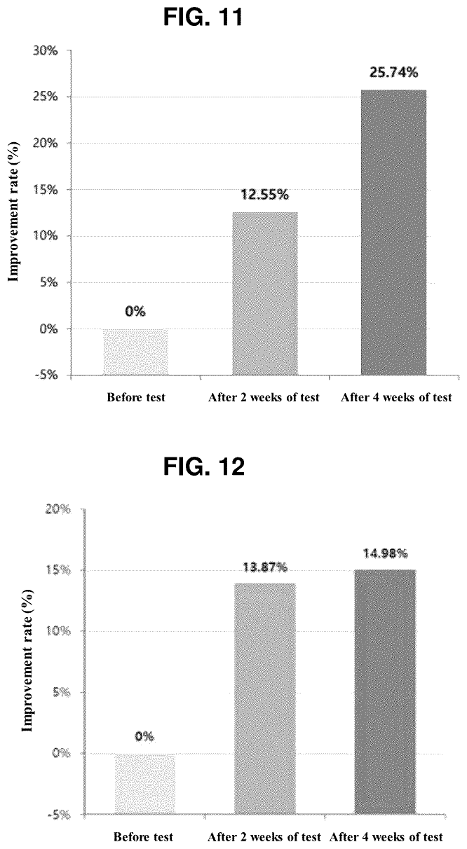

In particular, Example 4 showed the highest elasticity improvement rate of 25.74% (FIG. 11), which was about 2.7 times higher than that of Comparative Example 3 (9.65%).

The above results suggest that the fermentation product sample of the Example is effective in improving scalp elasticity, and in particular, these results show that the specific combination of the microorganisms mixed at the specific mixing ratio had a positive effect on restoring scalp elasticity.

### 5) Human Application Test for Improvement in Antioxidant Activity of Scalp Dead Skin Cells

The sample solution of each of the Examples and the Comparative Examples was applied to the scalp. After one month, the degree of improvement in the antioxidant activity of scalp-dead skin cells was evaluated.

Using Corneofix F20, dead skin cells were collected from a point approximately 15 cm away from the forehead on an imaginary line connecting the forehead and the crown of the head. The antioxidant activity (catalase activity) of the scalp dead skin cells was evaluated, and the rate of change in the antioxidant activity of the scalp dead skin cells was calculated.

TABLE 6

| | Rate (%) of improvement in |
|---|---|
| | antioxidant activity of scalp dead |
| | cells |
| Example 1 | 11.26 |
| Example 2 | 12.74 |
| Example 3 | 12.37 |
| Example 4 | 14.98 |
| Comparative Example 1 | 5.47 |
| Comparative Example 2 | 6.23 |
| Comparative Example 3 | 7.43 |
| Comparative Example 4 | 6.92 |

Referring to Table 6 above, the Examples generally showed a higher rate of improvement in the antioxidant activity of scalp dead skin cells than the Comparative Examples.

In particular, Example 4 showed the highest rate of improvement in antioxidant activity of scalp dead skin cells of 14.98% (FIG. 12), which was about 2.7 times higher than that of that of Comparative Example 1 (5.47%).

The above results suggest that the fermentation product sample of the Example can effectively improve the antioxidant activity (catalase activity) of scalp dead skin cells. In particular, these results show that the specific combination of the microorganisms mixed with the specific mixing ratio contributed to reducing the oxidative stress of scalp-dead skin cells and increasing the antioxidant defense ability thereof.

### 6) Human Application Test for Alleviation of Scalp Itching

The sample solution of each of the Examples and the Comparative Examples was applied to the scalp of experimental groups that experienced scalp itching due to UV radiation or a dry environment, and then the scalp was observed for one month.

The itching score (visual analog scale (VAS)) was used to evaluate changes in scalp itching.

The itching score was rated from 0 (no itching) to 10 (severe itching), and the alleviation effect was analyzed by comparing the itching scale between before the start of the experiment and after one month of the experiment. As a result value, the rate of increase in the itching score was calculated.

TABLE 7

| | Scalp itching alleviation rate |
|---|---|
| | (%, after 4 weeks) |
| Example 1 | 39.5 |
| Example 2 | 42.7 |
| Example 3 | 44.3 |
| Example 4 | 47.5 |
| Comparative Example 1 | 27.2 |
| Comparative Example 2 | 29.6 |
| Comparative Example 3 | 30.2 |
| Comparative Example 4 | 25.2 |

Referring to Table 7 above, the Examples generally showed a higher rate of alleviation of scalp itching than the Comparative Examples.

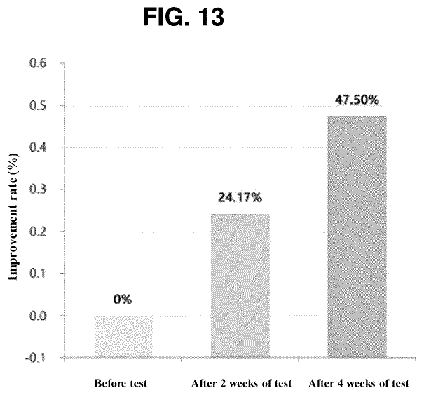

In particular, Example 4 showed the improvement rate of scalp itchiness in Example 4 was the highest at 47.5% (FIG. 13), which was about 1.9 times higher than that of Comparative Example 4 (25.2%).

The above results suggest that the fermentation product sample of the Example is effective in alleviating scalp itching caused by UV radiation and a dry environment.

### 7) Human Application Test for Scalp Microbiome Balance

5% culture medium composition containing the metabolites produced by culturing each of Lactococcus lactis, Limosilactobacillus reuteri, Saccharomyces cerevisiae, and Lactiplantibacillus plantarum was applied to the scalp of test subjects for 2 weeks. Thereafter, the scalp microbiome was compared between before and after application.

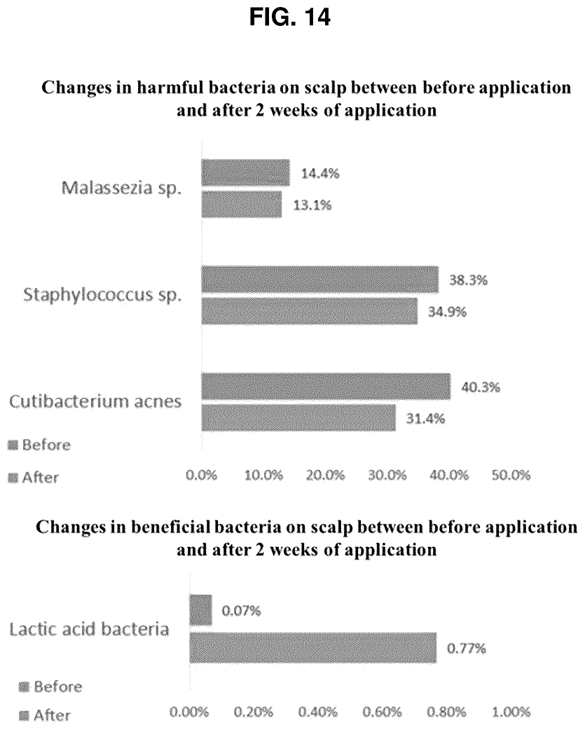

As a result, as shown in FIG. 14, it was confirmed that Malassezia sp., dandruff-causing bacteria, was reduced by 1.3%, Staphylococcus sp., scalp redness- and inflammation-causing bacteria, was reduced by 3.4%, and Cutibacterium acnes, scalp acne-causing bacteria, was reduced by 9%.

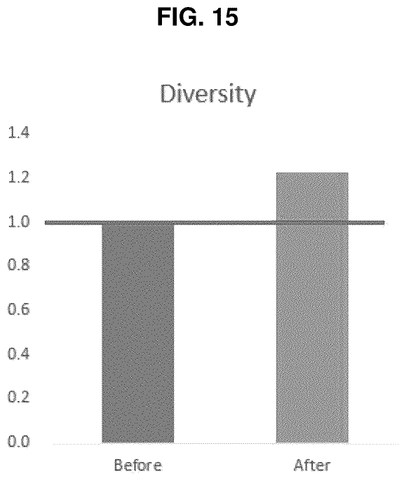

In addition, as shown in FIG. 15, the diversity of microorganisms on the scalp was increased by 20%.

The above experimental results above show that the specific combination of the microorganisms mixed at the specific mixing ratio Examples 1 to 4 exhibited a synergistic effect and exhibited a maximized effect of improving the scalp.

In particular, Example 4 exhibited an effect that was about twice higher than those of the Comparative Example in most evaluation items, including alleviation of itching, antioxidant activity, and pH normalization, suggesting that Example 4 amplified the effects of individual ingredients through interaction between the microorganisms.

The foregoing description of the present invention is provided by way of example, and it will be understood by those of ordinary skill in the art to which the invention pertains that various modifications can be made in other specific forms without departing from the technical spirit or essential characteristics of the present invention.

Therefore, the embodiments described above should be understood as illustrative in all respects and not limiting. For example, each component described as a single unit may be implemented in a distributed manner, and similarly, components described as being distributed may be implemented in a combined form.

The scope of the present invention is indicated by the claims provided below, and all modifications or variations derived from the meaning, scope, and equivalent concepts of the claims should be interpreted as being included within the scope of the present invention.

1. A cosmetic composition for improving the scalp containing a fermentation product of Lactococcus lactis and a fermentation product of Limosilactobacillus reuteri as active ingredients.

2. The cosmetic composition of claim 1, further containing a fermentation product of Saccharomyces cerevisiae as an active ingredient.

3. The cosmetic composition of claim 1, further containing a fermentation product of Lactiplantibacillus plantarum as an active ingredient.

4. The cosmetic composition of claim 1, wherein the fermentation products as the active ingredients comprise arginine, tocopherol, and proline.

5. The cosmetic composition of claim 1, which reduces scalp redness.

6. The cosmetic composition of claim 1, which removes scalp dead skin cells.

7. The cosmetic composition of claim 1, which improves scalp pH.

8. The cosmetic composition of claim 1, which alleviates scalp inflammation.

9. The cosmetic composition of claim 1, which improves scalp elasticity.

10. The cosmetic composition of claim 1, which improves the antioxidant activity of scalp dead skin cells.

11. The cosmetic composition of claim 1, which alleviates scalp itching.

12. A method for producing a cosmetic composition for improving the scalp, comprising:
step (a) of preparing a culture medium containing peptone, yeast, and a carbon source;
step (b) of producing a microbial fermentation product by culturing at least one of Lactococcus lactis, Limosilactobacillus reuteri, Saccharomyces cerevisiae, and Lactiplantibacillus plantarum in the culture medium; and
step (c) of mixing the culture medium and the microbial fermentation product, followed by culturing.
